(19) United States
(12) Patent Application Publication
(10) Pub. No.: US 2025/0352082 A1
(43) Pub. Date: Nov. 20, 2025

(54) METHOD AND SYSTEM OF QUANTIFYING LESION EVOLUTION

(71) Applicant: SyntheticMR AB (publ), Linköping (SE)

(72) Inventors: Johanna ALFREDSSON, Linköping (SE); Marcel WARNTJES, Linköping (SE)

(21) Appl. No.: 19/283,164

(22) Filed: Jul. 28, 2025

Related U.S. Application Data
(63) Continuation-in-part of application No. 17/187,281, filed on Feb. 26, 2021, now abandoned.

(51) Int. Cl. A61B 5/055; A61B 5/00; G01R 33/56; G06T 7/00; G06T 7/62; G06T 11/20; G16H 50/50

(52) U.S. Cl. CPC A61B 5/055; A61B 5/0042; A61B 5/4842; A61B 5/7425; G01R 33/5608; G06T 7/0016; G06T 7/62; G06T 11/203; G06T 11/206; G16H 50/50; A61B 2576/026; G06T 2200/24; G06T 2207/10088; G06T 2207/30012; G06T 2207/30016; G06T 2207/30096; G06T 2210/41

(57) ABSTRACT

A method of quantifying lesion evolution, comprising: acquiring, at a first time point, by a magnetic resonance, MR, scanning device, a single MR sequence of a portion of a body, the single MR sequence comprising quantitative information of the portion; generating, by a processing circuit, a first MR image representing the portion, based on the single MR sequence, wherein each voxel of the first MR image represents a corresponding volume of the portion; providing a lesion evolution model comprising at least two predetermined sets of quantitative values, wherein each of the at least two predetermined sets of quantitative values is associated with a reference lesion evolution value indicating a status of lesion evolution; for a voxel of a region of interest of the first MR image, determining, by the processing circuit, a lesion evolution value of said voxel, indicating a status of lesion evolution at the first time point.

This application is a continuation-in-part of U.S. application Ser. No. 17/187,281.

### TECHNICAL FIELD

The present document relates to a method and system of quantifying lesion evolution of a portion of a body. Particularly, the present document relates to a method and system of quantifying, visualizing a lesion evolution of a portion of a body, based on magnetic resonance imaging techniques.

### BACKGROUND

A lesion can be any damages or abnormal changes in a tissue of an organism, usually caused by a disease or trauma. Lesions can appear as a result of trauma or degenerative diseases. However, lesions can also indicate a region of a tumor. Lesions can exhibit different types of states, such as inflammation, edema, tissue destruction and necrosis. Lesions can occur anywhere in a body. Lesions may occur in humans, as well as other animals.

Lesions can be detected by different types of imaging techniques, including Magnetic Resonance Imaging (MRI).

A typical MRI scanner comprises a large, powerful magnet, and can send signals to, e.g., a body portion of a patient. The returning signals can be detected and converted into images of the body portion by a computing device. The images can be obtained in multiple planes (axial, sagittal, coronal, or oblique) without repositioning the patient.

The MRI techniques are generally based on relaxation properties of excited hydrogen nuclei (protons) of an object under test. When the object to be imaged is placed in a powerful, uniform magnetic field of the MRI scanner, the spins of the atomic nuclei of water in the object with non-integer spin numbers within the object all align either parallel or anti-parallel to the magnetic field. From an MR acquisition, several physical properties of the object under test can be determined. And an image can be reconstructed based on an acquired magnetic resonance sequence generated with the excitation.

Proton density (PD) refers to a concentration of protons in a tissue, wherein the protons are the hydrogen nuclei that resonate and give rise to the nuclear magnetic resonance signal. Since most visible tissue protons are resident in water, it is often seen as looking at a water content. The proton density PD of a tissue usually refers to the concentration of protons in the tissue, relative to that in the same volume of water at the same temperature.

The following time constants involved in the relaxation processes, which establish equilibrium following RF excitation, should be introduced in order to understand the MRI techniques. A nuclear magnetic resonance signal is affected by two simultaneous relaxation processes. The loss of coherence of the spin system attenuates the MRI signal with a time constant called a transverse relaxation time (T2). Concurrently, the magnetization vector slowly relaxes towards its equilibrium orientation that is parallel to the magnetic field by a time constant called longitudinal relaxation time (T1). A longitudinal relaxation rate R1 is the reciprocal of the longitudinal relaxation time T1 (R1=1/T1). A transverse relaxation rate R2 is the reciprocal of the transverse relaxation time T2 (R2−1/T2). The relaxation times T1 and T2 are typically measured in milliseconds (ms) or seconds(s). The corresponding relaxation rates R1 and R2 are therefore measured in units of ms⁻¹ or s⁻¹.

Normally, an acquired MRI sequence can result in images of the same anatomical section under different contrasts, such as T1-weighted, T2-weighted and PD-weighted images. The MRI techniques rely on differences in relaxation properties and proton density of the imaged tissue to display the different tissues with contrast, e.g., in different signal intensities or different colors, in the resulting MR images. The contrast in MR images originates from the fact that different tissues have, in general, different R1 and R2 relaxation rates, and different PD. For example, Warntjes, M. J. B. 1973, Dahlqvist, O. 1978, West, J., & Lundberg, P. 1958. (2008). Rapid magnetic resonance quantification on the brain: Optimization for clinical usage. Magnetic Resonance in Medicine, 60(2), 320-329 teaches that these physical properties, e.g., R1 and R2 relaxation rates and PD, can be acquired by performing a single MR acquisition, to provide quantitative values of the imaged portion.

Thus, using MRI technique, it is possible to generate various types of MR images, e.g., contrast weightings images which enhance and suppress different tissue types. By observing the MR contrast weighted images, e.g., the T1-weighted images, it is possible to get an idea of a status of an imaged portion.

However, even with the help of the MRI techniques, it is still difficult to accurately understand the status of a lesion describing how severe the tissue damage is, and/or how well the tissue recovers, as the grey scale MR images cannot provide too many details about the lesion evolution. Further, it is difficult to compare lesion statuses of different patients or patient groups, and/or of the same lesion over time in a quantitative way.

Thus, there is a need to provide a method and system to improve the quantifying, visualization and assessment of lesion evolution.

### SUMMARY

It is an object of the present disclosure, to provide a new method and system of quantifying lesion evolution of a portion, which eliminates or alleviates at least some of the disadvantages of the prior art.

The invention is defined by the appended independent claims. Embodiments are set forth in the appended dependent claims, and in the following description and drawings.

According to a first aspect, there is provided a method of quantifying lesion evolution, comprising: acquiring, at a first time point, by a magnetic resonance, MR, scanning device, a single MR sequence of a portion of a body, the single MR sequence comprising quantitative information of the portion; generating, by a processing circuit, a first MR image representing the portion, based on the single MR sequence, wherein each voxel of the first MR image represents a corresponding volume of the portion; providing a lesion evolution model, wherein the lesion evolution model comprises at least two predetermined sets of quantitative values, comprising a first redetermined set of quantitative values representing the portion at an initial status of lesion evolution, and a second predetermined set of quantitative values representing the portion at a final status of lesion evolution, and wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution; for a voxel of a region of interest of the first MR image, determining, by the processing circuit, a lesion evolution value of said voxel, indicating a status of lesion evolution at the first time point, based on a comparison between quantitative values of the voxel and the lesion evolution model.

The lesion may comprise a tissue abnormality.

The term “tissue abnormality” refers to any deviation from the normal structure, appearance, or function of tissue. This can include, but not limited to: tumors, lesions, inflammatory changes, fibrosis, calcifications, or other pathological alterations or non-pathological alternations.

Lesion evolution typically refers to change of a lesion, such as worsening or advancement of a lesion (e.g., an increase in size, number, or severity), over time. This can include: the growth/shrinkage of an existing lesion, the appearance of new lesions, the disappearance of an existing lesion, or a change in lesion characteristics, e.g., density, or shape.

In the field of medical imaging and diagnosis, lesion evolution is often used to evaluate the effectiveness of a treatment or diagnostic method. For example, new lesions or significant growth of existing lesions are considered indicators of disease advancement. In the present application, the terms lesion evolution and lesion evolution have the same meaning and are interchangeable.

Quantitative MRI (qMRI) is one division of the MRI techniques which measures absolute values, instead of measuring relative scales, of physical properties of a portion. The first/single MR sequence may be a quantitative MR sequence. By analyzing measured absolute values of certain physical properties of an imaged portion, it is possible to determine tissue composition of the imaged portion. Since the tissue composition normally changes along with the lesion evolution, e.g., due to inflammation and oedema, the tissue composition, and/or changes of the tissue composition, may be used to estimate and/or quantify lesion evolution of the imaged portion.

The tissue composition of the imaged portion may be estimated and/or quantified based on the measured absolute values of the physical properties. It may provide a fast and reliable tissue composition analysis of the imaged portion. The lesion evolution of the imaged portion may be estimated and/or quantified based on the determined tissue composition.

A lesion evolution value may indicate a status of lesion evolution, i.e. a severity of the lesion. For example, a low evolution value, such as 0, may resemble a healthy portion, a high evolution value, such as 100, may indicate tissue destructions and necrosis of the portion, and an evolution value in between, such as 1-99, may respectively indicate a unique status of lesion evolution between a healthy status to a necrosis status.

The method may provide a fast and reliable lesion evolution analysis of the imaged portion. It may facilitate monitoring of the lesion evolution or recovery over time. It may facilitate comparison of lesion evolution between portions of different objects, e.g., different patients. A quantified lesion evolution may be readily visualized.

Voxels are frequently used in the visualization and analysis of medical 3D images. A voxel is a volume element, used to represent a tiny 3D volume in an imaged portion. Here, each voxel of the MR image represents a corresponding tiny volume of the imaged portion. Thus, each voxel may have quantitative values, e.g., R1 and R2, representing physical properties of the corresponding tiny volume of the imaged portion.

A pixel is an element, used to represent a tiny 2D part in a 2D image. The 3D imaged portion may be sliced into a stack of slices each having a thickness. A voxel may be considered to correspond to a pixel for a given slice thickness. In other words, a voxel can be considered as a volumetric pixel for the given slice thickness. Thus, a 3D image may be converted into a series of 2D images. Consequently, the voxels may be converted into a series of pixels.

The step of acquiring the MR sequence and the step of generating the MR image may be one step instead of two. It is common that an MR scanner may perform an acquisition and result in one or more MR images representing a layer of the imaged portion.

The portion of the body may comprise a head or any part of a spine.

The quantitative information may comprise information of at least two among the physical properties: a longitudinal relaxation rate R1, a transverse relaxation rate R2, a longitudinal relaxation time T1, a transverse relaxation time T2, and a Proton Density, PD.

Using a combination of at least two different physical properties, the tissue composition can be determined.

The physical properties R1 and/or R2 may be replaced by the physical properties longitudinal relaxation time T1 and transverse relaxation time T2, respectively.

The step of determining a lesion evolution value of said voxel may comprise: among the at least two predetermined sets of quantitative values of the lesion evolution model, determining a set of quantitative values being closest to the quantitative values of the voxel, and determining the lesion evolution value of the voxel to be equal to the reference lesion evolution value associated with the determined set of quantitative values.

The term “closest” may refer to one set of quantitative values being the same as another set of quantitative values.

Alternatively, the term “closest” may refer to one set of quantitative values having a least difference, i.e. a least deviation, from another set of quantitative values. There are many known mathematical methods for comparing two sets of values and determining a difference between them.

The method may comprise for at least one of the at least two predetermined sets of quantitative values, calculating the reference lesion evolution value to be associated, based on said predetermined set of quantitative values.

Said predetermined set of quantitative values may comprise a value of the longitudinal relaxation rate R1 and a value of the transverse relaxation rate R2.

The method may further comprise: calculating the lesion evolution value to be associated by Lesion Evolution Value=norm(R1)*norm(R2),

wherein Lesion Evolution Value refers to the reference lesion evolution value to be associated with said set of quantitative values, R1 and R2 respectively refer to the values of the longitudinal relaxation rate R1 and the transverse relaxation rate R2 of said set of quantitative values, norm refers to a norm function.

The above equation is an example of how to determine the lesion evolution value based on the set of quantitative values.

The method may comprise scatter-plotting the at least two predetermined sets of quantitative values into at least two lesion evolution points, respectively, in a coordinate system, wherein the first and second predetermined set of quantitative values are scatter-plotted into a first lesion evolution point and a second lesion evolution point, respectively.

The coordinate system may be a Cartesian coordinate system. The coordinate system may be of different dimensions, such as two dimensions or three dimensions, depending on the number of values of each set of quantitative values. For example, if quantitative information comprises information of two physical properties: R1 and R2, then the coordinate system may be a two-dimensional coordinate system comprising an x-axis and a y-axis representing the values of R1 and R2, respectively.

The method may comprise generating a lesion evolution curve generating a lesion evolution curve passing through the at least two lesion evolution points one by one in the coordinate system, following an order of lesion evolution from the initial status to the final status, wherein the lesion evolution curve starts from the first lesion evolution point, and ends at the second lesion evolution point, or following an order of lesion evolution from the final status to the initial status, wherein the lesion evolution curve starts from the second lesion evolution point, and ends at the first lesion evolution point.

The lesion evolution curve may be generated by connecting the lesion evolution points one by one in the coordinate system. The lesion evolution curve may be continuous or comprising at least two discontinuous portions.

The lesion evolution curve may comprise a straight portion.

For example, the first lesion evolution point may refer to a healthy portion and the second lesion evolution point may refer to a necrotic portion. Alternatively, the first lesion evolution point may refer to a necrotic portion and the second lesion evolution point may refer to a healthy portion.

The method may comprise generating a parametric representation describing a relationship between the at least two lesion evolution points in the coordinate system, based on the at least two predetermined sets of quantitative values.

The parametric representation may be a parametric equation defining a group of quantities as functions of independent parameters. Parametric equations are commonly used to express points making up a curve or surface of a coordinate. For example, the parametric representation may be a polynomial function describing a relationship of the at least two lesion evolution points in the coordinate system, based on the at least two sets of quantitative values.

The method may comprise creating a predetermined number of new lesion evolution points between the first and the second lesion evolution point in the coordinate system, based on the generated lesion evolution curve or the generated parametric representation, wherein each of the new lesion evolution points may correspond to a new set of quantitative values.

The new lesion evolution points may be generated by interpolation.

The method may comprise for each new set of quantitative values, associating a new reference lesion evolution value indicating a new status of lesion evolution.

For each new set of quantitative values, the associated new reference lesion evolution value may be determined based on the generated lesion evolution curve or the generated parametric representation, and the at least two predetermined sets of quantitative values and their associated reference lesion evolution values.

The lesion evolution value, the lesion evolution point, and the set of quantitative values may have a one-to-one corresponding relationship. That is, by knowing the quantitative values of a voxel representing a corresponding volume of a portion, the voxel's lesion evolution value for indicating the status of lesion evolution of the corresponding volume can be determined.

In other words, the lesion evolution points in the coordinate system may link the lesion evolution values of a voxel with the quantitative values of said voxel.

For a new lesion evolution point positioned between the first and second lesion evolution point, its associated new lesion evolution value may be determined by the position of the new lesion evolution point along the lesion evolution curve.

For example, if the new lesion evolution point is positioned in the middle point along the lesion evolution curve between the first and second lesion evolution point, its associated new lesion evolution value may be an average of the first and second lesion evolution value.

The method may comprise generating a new lesion evolution curve or refining an existing lesion evolution curve in the coordinate system, which passes through the at least two lesion evolution points and the generated new lesion evolution points one by one, following an order of lesion evolution from the initial status to the final status, wherein the generated new lesion evolution curve or the refined lesion evolution curve starts from the first lesion evolution point, and ends at the second lesion evolution point, or following an order of lesion evolution from the final status to the initial status, wherein the generated new lesion evolution curve or the refined lesion evolution curve starts from the second lesion evolution point, and ends at the first lesion evolution point.

For example, the first lesion evolution value is 0 and the second lesion evolution value is 4, and there are three newly created lesion evolution points sequentially positioned between the first and the second lesion evolution points based on the parametric representation. These three new lesion evolution points may be used to refine the already generated lesion evolution curve. The associated new lesion evolution values, e.g., 0.5, 2.5 and 3, for the three new set of values of quantitative information may be determined based on the parametric representation, and the first and the second lesion evolution value of the end points of the lesion evolution curve.

The new lesion evolution points and the at least two lesion evolution points may be evenly positioned along the generated new lesion evolution curve or the refined lesion evolution curve.

The associated reference lesion evolution values of the at least two predetermined sets of quantitative values and of the new sets of quantitative values may form an arithmetic evolution; wherein the arithmetic evolution may start with the first reference lesion evolution value and end with the second reference lesion evolution value; and wherein a position of each reference lesion evolution value of the arithmetic evolution may correspond to a position of its corresponding lesion evolution point along the generated new lesion evolution curve or the refined lesion evolution curve.

An arithmetic evolution is a sequence of numbers, wherein a difference between any two consecutive numbers is constant.

For example, the first reference lesion evolution value is 0 and the second reference lesion evolution value is 100, and there are nine new lesion evolution points sequentially positioned between the first and the second lesion evolution points along the lesion evolution curve. Then, the newly created lesion evolution point positioned immediately adjacent to the first lesion evolution point may have a reference lesion evolution value “10”, its next lesion evolution point may have a reference lesion evolution value “20”, . . . , and the nineth new lesion evolution point positioned immediately adjacent to the second lesion evolution point may have a reference lesion evolution value “90”. In this example, the reference lesion evolution values of the lesion evolution points form an arithmetic evolution (0, 10, 20, . . . , 90, 100), regardless of whether the new lesion evolution points are arranged evenly in between the first and second lesion evolution point.

The term “evenly” may refer to a fixed distance between any two adjacent lesion evolution points along the lesion evolution curve. As the previous example, if the nine new lesion evolution points are evenly and sequentially positioned between the first and the second lesion evolution points along the lesion evolution curve, the lesion evolution values of these nine lesion evolution points together with the first reference lesion evolution value 0 and the second reference lesion evolution value 100, may form an arithmetic evolution having 11 numbers, starts with 0 and ends with 100. Consequently, the nine reference lesion evolution values of the nine new lesion evolution points are respectively 10, 20, . . . , 80 and 90. That is, the arithmetic evolution is 0, 10, 20, . . . , 80, 90, 100.

The initial status may be a normal status and the final status may be an abnormal status. Alternatively, the initial status may be an abnormal status and the final status may be a normal status.

The normal status may refer to a healthy status of a portion. The abnormal status may refer to an unhealthy status, including but not limited to an inflammation status, an oedema status, or a necrosis status.

The lesion evolution model may cover an entire lesion evolution from a healthy status to a necrosis status, or from the necrosis status to the healthy status. Alternatively, the lesion evolution model may cover only a part of the entire lesion evolution.

The method may comprise visualizing lesion evolution of the portion, comprising: displaying, by a user interface, the determined lesion evolution value.

The method may comprise repeating the step of determining a lesion evolution value for each voxel of the region of interest. That is, the status of lesion evolution of any volume corresponding to a voxel of the region of interest may be determined.

The region of interest may be a part of the first MR image or the entire first MR image.

An average lesion evolution value of the region of interest may be determined. This average lesion evolution value may indicate an average status of lesion evolution of a portion corresponding to the region of interest of the first MR image. Thus, the status of lesion evolution of the region of interest may be represented by one value, which can simplify the representation of the lesion status. That is, the lesion status of the portion may be visualized by one or more numerical values instead of any images, e.g., the MR images with an overlay in FIG. 4C.

Even though the numerical representation of the lesion status of the portion may be considered simple, it is a powerful tool in many scenarios. For example, the numerical representation can be used to provide a fast comparison between different patient groups, and/or between a patient group and its healthy peers. Further, the numerical representations of a same patient may change over time and from scan to scan, which can be used to monitor the lesion status development and recovery process of a patient.

The average values calculated for one or more regions of the MR image may be displayed and/or output as a table, e.g., for recording and reporting.

The method may comprise for each determined lesion evolution value, calculating a volume size of a partial portion having the status of lesion evolution indicated by said determined lesion evolution value; and displaying a distribution of determined lesion evolution values for each voxel of the region of interest in a form of histogram.

The histogram may be a lesion evolution profile in a coordinate system. An x-axis of the coordinate system may represent lesion evolution values, and a y-axis of the coordinate system may represent a volume size for each lesion evolution value or lesion evolution value range.

It is possible to determine an absolute volume size, e.g., in the unit of ml, of a partial portion of a tissue corresponding to the region of interest of the MR image, having said determined status of lesion evolution.

Alternatively, the volume size may be a percentage value, indicating a percentage of the partial portion having said determined status of lesion evolution.

This may provide additional valuable information about the tissue corresponding to the region of interest of the MR image, e.g., exactly how much tissue exhibits a status of necrosis within the tissue.

The method may comprise displaying a distribution of determined lesion evolution values for each voxel of the region of interest. The distribution may in the form of histogram or a table of numbers.

The distribution of the lesion evolution values may provide a fast and straightforward view of the overall status of the tissue.

The distribution of the lesion evolution values can be used for monitoring the lesion evolution of one patient over time or comparing the lesion evolutions of different patients. It is advantageous as it is much easier to perform a comparison between two distributions than performing a comparison between two MR images.

The method may further comprise: visualizing lesion evolution of the portion, comprising: displaying the first MR image, wherein the voxels of the region of interest may be displayed differently, based on their respective lesion evolution value.

The voxels of the region of interest may be displayed with different colors and/or different intensities based on their respective lesion evolution values.

For example, a voxel having a high lesion evolution value may be displayed in a high intensity and/or a bright color, and another voxel having a low lesion evolution value may be displayed in a low brightness and/or a dark color.

Colors and/or intensities may visualize the status of lesion evolution.

The term “intensity”, also known as “signal intensity”, in the field of MR may refer to a shade of grey of a tissue or of a voxel representing the tissue in an MR image. Generally, a high intensity means it would look “white/bright” in the MR image, an intermediate intensity means it would look “grey” in the MR image, and a low intensity means it would look “black” in the MR image.

The method may further comprise: visualizing lesion evolution of the portion, comprising: displaying the first MR image or a different MR image representing the portion as a background image; and displaying an overlay to the background image. Voxels of the overlay corresponding to the voxels of the region of interest may be displayed differently, based on the lesion evolution values of the voxels of the region of interest.

The voxels displayed differently may be displayed in different colors and/or intensities.

The different MR image may be an MR image without any quantitative information. For example, the different MR image may be a T1-weighted (T1W) image.

By displaying the background image representing an anatomy of the imaged portion, a user may easily correlate a lesion evolution value to an original anatomy of the lesion portion, such that the assessment of the lesion can be facilitated.

The method may comprise displaying a color scale as a reference for visualizing the lesion evolution value for each displayed color of the voxels displayed differently; and/or displaying an intensity scale as a reference for visualizing the lesion evolution value for each displayed intensity of the voxels displayed differently.

The method may comprise acquiring, at a second time point, a second single MR sequence of the portion comprising quantitative information of the portion; generating a second MR image representing the portion, based on the second single MR sequence, wherein the second MR image comprises a second region of interest, wherein both the region of interest and the second region of interest correspond to a same part of the portion; for a voxel of the second region of interest, determining a second lesion evolution value, based on quantitative values of said voxel of the second region of interest and the lesion evolution model; and determining a change of lesion evolution of the portion from the first time point to the second time point, based on the determined second lesion evolution value, and the lesion evolution value of its corresponding voxel of the region of interest.

This may be advantageous as it is possible to compare the lesion evolution of a same lesion over time for a patient. This may facilitate a better monitoring of the changes of lesions. This may be used for predicting further lesion changes, such as the tissue destruction progress.

The step of determining a change of lesion evolution of the portion from the first time point to the second time point may comprise: repeating the step of determining a lesion evolution value for each voxel of the region of interest; for each determined lesion evolution value, calculating a volume size of a partial portion having the status of lesion evolution indicated by said determined lesion evolution value; displaying a distribution of determined lesion evolution values for each voxel of the region of interest as a first lesion evolution profile in a coordinate system; wherein an x-axis of the coordinate system represents lesion evolution values, and a y-axis of the coordinate system represents a volume size for each lesion evolution value or lesion evolution value range; repeating the step of determining a second lesion evolution value for each voxel of the second region of interest; for each determined second lesion evolution value, calculating a volume size of a partial portion having the status of lesion evolution indicated by said determined second lesion evolution value; displaying a distribution of determined lesion evolution values for each voxel of the region of interest as a second lesion evolution profile in the coordinate system; displaying the change of lesion evolution of the portion from the first time point to the second time point by subtracting the first lesion evolution profile from the second lesion evolution profile.

The portion of the body may comprise a head or any part of a spine.

According to a second aspect, there is provided a system for quantifying lesion evolution, comprising a magnetic resonance, MR, scanning device configured to: acquire, at a first time point, a single MR sequence of a portion of a body, the single MR sequence comprising quantitative information of the portion; the system for quantifying lesion evolution comprising a processing circuit configured to: generate a first MR image representing the portion, based on the single MR sequence, wherein each voxel of the first MR image represents a corresponding volume of the portion; and determine a lesion evolution value indicating a status of lesion evolution for a voxel of a region of interest of the first MR image, based on a comparison between quantitative values of the voxel and a lesion evolution model; wherein the lesion evolution model comprises at least two predetermined sets of quantitative values, comprising a first predetermined set of quantitative values representing the portion at an initial status of lesion evolution, and a second predetermined set of quantitative values representing the portion at a final status of lesion evolution; wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution.

The system may further comprise a user interface configured to display information for visualizing lesion evolution of the portion.

The user interface may be configured to output information, such as texts, sounds, images, etc.

The user interface may be configured to receive input, e.g., a command, from a terminal or an input device via a wire or wirelessly. The user interface may be configured to receive input from a user.

According to a third aspect, there is provided a non-transitory computer readable recording medium having computer readable program code recorded thereon which when executed on a device having processing capability is configured to perform the method of the first aspect.

### BRIEF DESCRIPTION OF THE DRAWINGS

FIG. 1 is an example plot of quantitative values of healthy and pathological tissue.

FIGS. 2A-2B are MR images visualizing lesions of different statuses.

FIG. 3 is an example of migration patterns.

FIGS. 4A-4C are images for visualizing lesion.

FIGS. 5-6 are examples of visualized lesion evolution status.

FIGS. 7A-7B visualize correlation between lesion evolution value and myelin content.

FIGS. 8A-8B visualize correlation between lesion evolution value and excess water content.

FIGS. 9A-9B visualize correlation between lesion evolution value and T1 signal intensity.

FIGS. 10A-10B visualize correlation between lesion evolution value and T2FLAIR signal intensity.

FIG. 11 is an example of a schematic block diagram of a system for quantifying lesion evolution.

FIG. 12 illustrates exemplary evolution profiles.

### DESCRIPTION OF EMBODIMENTS

The present invention will now be described more fully with reference to the accompanying drawings, in which currently preferred embodiments of the invention are shown.

In connection with FIG. 11, the system 1 for quantifying lesion evolution will be discussed in more detail.

The system 1 comprises a processing circuit 3. The processing circuit 3 is configured to carry out overall control of functions and operations of the system 1. The processing circuit 3 may include a processor, such as a central processing unit (CPU), microcontroller, or microprocessor. The system 1 may comprise a memory. The processing circuit 3 may be configured to execute program codes stored in the memory, in order to carry out functions and operations of the system 1.

The memory may be one or more of a buffer, a flash memory, a hard drive, a removable medium, a volatile memory, a non-volatile memory, a random access memory (RAM), or another suitable device. In a typical arrangement, the memory may include a non-volatile memory for long term data storage and a volatile memory that functions as system memory for the system 1. The memory may exchange data with the processing circuit 3 over a data bus. Accompanying control lines and an address bus between the memory and the processing circuit 3 also may be present.

Functions and operations of the system 1 may be embodied in the form of executable logic routines (e.g., lines of code, software programs, etc.) that are stored on a non-transitory computer readable medium (e.g., the memory) of the system 1 and are executed by the processing circuit 3. Furthermore, the functions and operations of the system 1 may be a stand-alone software application or form a part of a software application that carries out additional tasks related to the system 1. The described functions and operations may be considered a method that the corresponding device is configured to carry out. Also, while the described functions and operations may be implemented in software, such functionality may as well be carried out via dedicated hardware or firmware, or some combination of hardware, firmware and/or software.

The system 1 may comprise an MR scanning device 2. The MR scanning device 2 may be configured to acquire a single or multiple MR sequences of a portion comprising a head or any part of a spine. A plurality of slices may be generated based on the MR sequence of the portion, wherein each slice represents a layer of the portion.

The processing circuit 3 is configured to: generate a first MR image representing the portion, based on the single MR sequence, wherein each voxel of the first MR image represents a corresponding volume of the portion; and determine a lesion evolution value indicating a status of lesion evolution for a voxel of a region of interest of the first MR image, based on a comparison between quantitative values of the voxel and a lesion evolution model.

The lesion evolution model comprises at least two predetermined sets of quantitative values, comprising a first predetermined set of quantitative values representing the portion at an initial status of lesion evolution, and a second predetermined set of quantitative values representing the portion at a final status of lesion evolution; wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution.

The system 1 may comprise a user interface 4. The user interface 4 may be configured to output data and information, e.g., determined lesion evolution values, a first and second MR image. The user interface 4 may be configured to receive data and information, such as a command, from one or several input devices. The input device may be a computer mouse, a keyboard, a track ball, a touch screen, or any other input device. The user interface 4 may send the received data and information to the processing circuit 3 for further processing.

The method of quantifying lesion evolution comprises acquiring, at a first time point, by an MR scanning device 2, a single magnetic resonance, MR, sequence of a portion of a body, the single MR sequence comprising quantitative information of the portion; generating, by a processing circuit 3, a first MR image representing the portion, based on the single MR sequence, wherein each voxel of the first MR image represents a corresponding volume of the portion.

It is known that conventional imaging techniques, e.g., conventional MRI techniques, can generate images of a relative scale, such as contrast weighted images of a relative intensity scale. However, quantitative MRI (qMRI) techniques can measure absolute values of physical properties of observed tissues of the imaged portion. That is, a quantitative MRI acquisition may generate an MR sequence comprising quantitative information of the observed tissue. An MR image may be generated to represent the observed tissue, based on the MR sequence. The acquisition of the MR sequence and generation of the MR image may be two independent steps or combined as one step.

The application uses Multiple Sclerosis (MS) as the example for illustrating how the invention works. However, other diseases are analogously applicable.

Multiple Sclerosis (MS) is a well-known autoimmune disease and is characterized by focal areas of demyelination, inflammation, axonal loss and gliosis in neurological tissue. These changes of tissue occur dynamically, and not in any typical order.

The MS lesions may be in a head or in a spine.

Typically, the lesion will progress over time. Lesion evolution may represent the change of lesion tissue over time, e.g., from an initial status to a final status. The initial status and the final status may be defined differently, e.g., covering an entire lesion progress or a partial part of the entire lesion progress.

The lesion may progress or recover, e.g., by remyelination, to a healthy status. If no recovery occurs, the lesion may undergo necrosis, and an expansion of extracellular space may form, caused by tissue destruction which would cause extracellular free water to accumulate within the lesion tissue.

The changes of tissues may to some extent be observed in the contrast weighted MRI images generated by the conventional MRI techniques without quantitative information. For example, the MS lesion can be detected in a T2-weighted MR image by their hyperintense appearance. The MS lesions may shrink in size and become less hyperintense over time due to remyelination and decreasing edema. During active inflammation, most MS lesions appear hypointense in the T1-weighted image, but some may appear isointense. The hypointense appearance in this phase is a sign of inflammation, edema, demyelination and glial activation. Progressive repair processes including remyelination and resorption may change the tissue composition, causing the lesion to appear isointense again. However, some portion of the lesions may evolve to persistent black holes which is associated with permanent demyelination and severe axonal loss with irreversible tissue destruction and necrosis. The relevant information can be seen in e.g., Àlex Rovira, Cristina Auger, & Juli Alonso. (2013). Magnetic resonance monitoring of lesion evolution in multiple sclerosis. Therapeutic Advances in Neurological Disorders, 6, and Sahraian, M. A., Radue, E.-W., Haller, S., & Kappos, L. (2010). Black holes in multiple sclerosis: definition, evolution, and clinical correlations. Acta Neurologica Scandinavica, 122(1), 1-8.

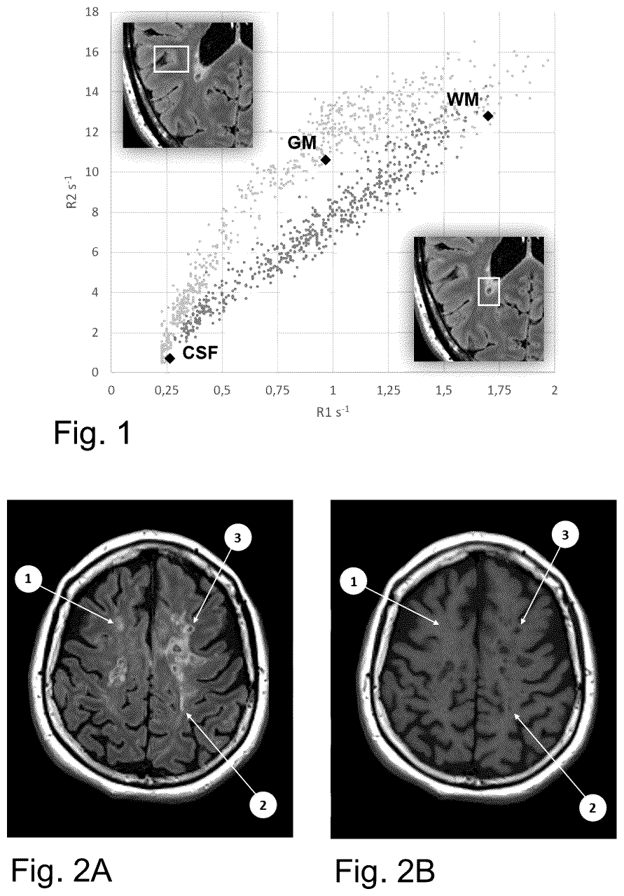

In connection with FIGS. 2A and 2B, different statuses of lesion evolution will be discussed in more detail.

FIGS. 2A and 2B are respectively a T2-weighted-Fluid-attenuated inversion recovery (T2FLAIR) image and a T1-weighted (T1W) image of a same patient.

The T2FLAIR image is based on an MR sequence with an inversion recovery set to null fluids. When an MS lesion suffers from severe tissue destruction, it may generate a hypointense signal in the T2FLAIR image. This is caused by the increased water content, which is suppressed by the inversion pulse in the Fluid-attenuated inversion recovery (FLAIR) setting. In other words, when the imaged portion is the brain, the T2FLAIR image can highlight MS lesions by suppressing CSF effects on the image. Thus, in T2FLAIR images, such as FIG. 2A, lesions are hyperintense (bright) comparing to its surrounding tissues. Thus, the hyperintense appearance in T2FLAIR images can be considered as a sign of inflammation, edema or axonal loss. However, it is not possible to differentiate these conditions based on the T2FLAIR images alone.

In the T1W images, such as FIG. 2B, the MS lesion in the early stages is isointense compared to its surrounding white matter (WM) and is therefore not detectable. When the MS lesion further progresses, a hypointense (dark) signal will be seen because of its increased water content, which is associated to axonal loss and edema. The hypointense lesions are often referred to as black holes and show signs of irreversible tissue destruction.

Three MS lesions of different lesion statuses are respectively marked as 1, 2 and 3 in FIGS. 2A and 2B. The no. 1 lesion is of an early stage of the lesion evolution, which has a vague appearance in FIG. 2A, and is invisible in FIG. 2B. The no. 2 lesion is of a progressed stage of the lesion evolution, which has an increase of edema and inflammation. The no. 2 lesion is clearly visible in both FIG. 2A and FIG. 2B. The no. 3 lesion is of a severe stage of the lesion evolution. In FIG. 2A, the no. 3 lesion is clearly visible, it can be seen that the center of the no. 3 lesion has a clear loss of signal due to the inversion pulse which suppress signal generated by in its increased fluid (water) content. In FIG. 2B, the no. 3 lesion has a distinct appearance indicating a black hole and severe axonal damage.

Both the T2FLAIR images and the T1W images generated by the conventional MRI techniques cannot give any sufficient information of an exact lesion status. In other words, it is not possible to fully understand the severeness of inflammation and destruction of a lesion tissue from the conventional contrast weightings.

In connection with FIG. 1, tissues and quantitative values of the voxels corresponding to the tissues will be discussed in more detail.

It is known that the qMRI techniques can measure absolute values of different physical properties of the imaged portion, e.g., the longitudinal relaxation rate R1, the transverse relaxation rate R2, and the proton density PD. The relaxation rates R1 and R2 are the respective inverse of the relaxation times T1 and T2, i.e. R1=1/T1 and R2=1/T2. The absolute values of physical properties measured by the qMRI techniques are independent of the MR scanning device's settings, and inhomogeneities of the magnetic field.

Different tissue types, such as white matter (WM), gray matter (GM) and cerebrospinal fluid (CSF) of the brain tissues, normally have different absolute values of physical properties, as seen in West, J. 1982, Warntjes, M. 1973, & Lundberg, P. 1958. (2012). Novel whole brain segmentation and volume estimation using quantitative MRI. European Radiology, 22(5), 998-1007. Table. 1 is an example of R1, R2 and PD values of WM, GM and CSF measured at 1.5 T.

TABLE 1
Typical values of physical properties of different tissue types at 1.5 T.

| | R1 s⁻¹ | R2 s⁻¹ | PD %⁻¹ |
|---|---|---|---|
| WM | 1.7 | 12.8 | 67.4 |
| GM | 0.967 | 10.6 | 86.9 |
| CSF | 0.266 | 0.705 | 103 |

Based on the measured absolute values of at least two physical properties, a tissue composition of the imaged tissue may be determined. For example, if the values of R1 and PD of one tissue are 1.7 s⁻¹ and 67%, respectively, the imaged tissue is determined to be WM, based on Table 1.

Consequently, when inflammation, edema and tissue destruction happen, due to the increased water content, the R1 and R2 values of the tissue may decrease and the PD value of the tissue may increase. When the destruction is severe, an extracellular space may be widened and filled by water. Then the absolute values of R1, R2 and PD of the imaged tissue may be similar to that of the CSF in Table 1, due to the large amount of water.

FIG. 1 is an example plot of R1 and R2 values of tissues in a two-dimensional (2D) space (R1-R2 space), such as a 2D Cartesian space or a 2D Cartesian coordinate.

An MR image representing a portion of a brain of a patient is respectively shown in the upper left corner and bottom right corner of FIG. 1. One healthy region of interest marked by a square representing a healthy brain portion is marked by a square in the upper left MR image. Another lesion region of interest marked by a square representing a brain portion of MS lesion (comprising both healthy brain tissues and MS lesion tissues) is marked by a square in the bottom right MR image.

Each data point in the plot of FIG. 1 is projected by the R1 and R2 values of a voxel of the healthy and lesion region of interest of the respective MR image. In other words, each data point of the plot represents a combination of the quantitative values of its corresponding voxel of the healthy and lesion region of interest of the MR image.

The data points represented the voxels of the healthy region of interest and the voxels of the lesion region of interest are displayed in the plot of FIG. 1 in different greyscales. The brighter data points represent the voxels of the healthy region of interest, and the darker data points represent the voxels of the lesion region of interest.

In the plot of FIG. 1, different clusters of data points are formed, as in West, J. 1982, Warntjes, M. 1973, & Lundberg, P. 1958. (2012). Novel whole brain segmentation and volume estimation using quantitative MRI. European Radiology, 22(5), 998-1007. That is, the data points representing voxels having similar values of physical properties are located close to each other as one cluster of data points. One cluster of data points may have the same tissue type different from another cluster of data points. The tissue type of one cluster can be identified based on the known values of physical properties, such as R1, R2 and PD values in Table 1. The example data points from the clusters corresponding to the tissue types of WM, GM and CSF are marked in the plot of FIG. 1.

In the plot of FIG. 1, it can be seen that the healthy brain portion (brighter data points) form clearly WM and GM clusters in the R1-R2 space. There are data points between the WM and GM clusters, which represent voxels of a mixture of WM and GM. Thus, the brighter data points of the plot of FIG. 1 can be considered as a typical R1-R2 pattern of a healthy brain parenchyma.

When the MS lesion is formed, e.g., due to demyelination, inflammation and edema, changes of the composition of the healthy brain tissues may happen. Consequently, the imaged portion comprising MS lesions may present a different pattern, e.g., a R1-R2 pattern, different from the typical pattern of the healthy brain parenchyma.

Such a different pattern can be called a migration pattern. The migration pattern visualizes the changes in the quantitative values that occur during the different stages of the lesion evolution. Comparing to the brighter data points, the darker data points form the migration patten of a reduced R1 and/or R2 values.

In the lesion region of interest, a faint hyperintense (bright) area representing the lesion tissue can be seen. Moving towards the center of the hyperintense area, the signal intensity is increased, and the R1 and R2 values of the voxels are further decreased. A necrotic core can be seen in the center of the lesion, which causes an influx of free water, resulting the quantitative values of the voxels very close to that of the CSF in Table 1.

Some of the brighter and darker data points overlap in the plot of FIG. 1. This is because the MS lesion brain portion also comprise healthy brain tissues, which would have quantitative values similar to that of the brighter data points.

The quantitative information may comprise information of at least two among physical properties: a longitudinal relaxation rate R1, a transverse relaxation rate R2, a longitudinal relaxation time T1, a transverse relaxation time T2, and a Proton Density, PD. The physical properties R1, R2 can be replaced by T1, T2, respectively. That is, plotting R1 and R2 values as in FIG. 1 is only one example. It is possible to use a combination of R1 and PD values, or a combination of R2 and PD values.

A combination of more than two physical properties may be used. For example, values of more than two physical properties may be plotted in a multiple-dimensional space. For example, if the quantitative information comprises information of R1, R2 and PD, a three-dimensional (3D) space (R1-R2-PD space) may be used to plot the R1, R2 and PD values.

The plot can be used for facilitating analysis of the imaged portion. However, the analysis can be performed merely based on quantitative information of the portion, without using any plots.

The method of quantifying lesion evolution comprises providing a lesion evolution model. The lesion evolution model comprises at least two predetermined sets of quantitative values, comprising a first redetermined set of quantitative values representing the portion at an initial status of lesion evolution, and a second predetermined set of quantitative values representing the portion at a final status of lesion evolution, and wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution. The method of quantifying lesion evolution comprises for a voxel of a region of interest of the first MR image, determining, by the processing circuit, a lesion evolution value of said voxel, indicating a status of lesion evolution at the first time point, based on a comparison between quantitative values of the voxel and the lesion evolution model.

The step of determining a lesion evolution value of said voxel may comprise: among the at least two predetermined sets of quantitative values of the lesion evolution model, determining a set of quantitative values being closest to the quantitative values of the voxel, and determining the lesion evolution value of the voxel to be equal to the reference lesion evolution value associated with the determined set of quantitative values.

If the lesion evolution model comprises only the first and second predetermined set of quantitative values, the determined lesion evolution value is rough and less accurate.

A more accurate estimation of lesion evolution status may be achieved if the lesion evolution model comprises a large number of predetermined sets of quantitative values. For each predetermined set of quantitative values, there may be a reference lesion evolution value indicating the status of lesion evolution associated with the predetermined set of quantitative values.

The reference lesion evolution value to be associated may be calculated based on said predetermined set of quantitative values.

For example, when a predetermined set of quantitative values comprises a value of the longitudinal relaxation rate R1 and a value of the transverse relaxation rate R2, the lesion evolution value to be associated may be calculated by these two values R1 and R2.

One example is

$$Lesion Evolution Value = norm ( R 1 ) * norm ( R 2 ) .$$

Lesion Evolution Value refers to the reference lesion evolution value to be associated with said predetermined set of quantitative values, and norm refers to a norm function.

The above equation is an example of how to determine the reference lesion evolution value based on the predetermined set of quantitative values. For example, the first reference lesion evolution value indicating the initial status of lesion evolution associated with the first set of quantitative values may be calculated, and/or the second lesion evolution value indicating the final status of lesion evolution associated with the second set of quantitative values may be calculated.

The lesion evolution values associated with other sets of quantitative values may be calculated by the same equation. Alternatively, they can be determined by known mathematical methods, based on the calculated lesion evolution value and the sets of predetermined quantitative values, such as interpolation.

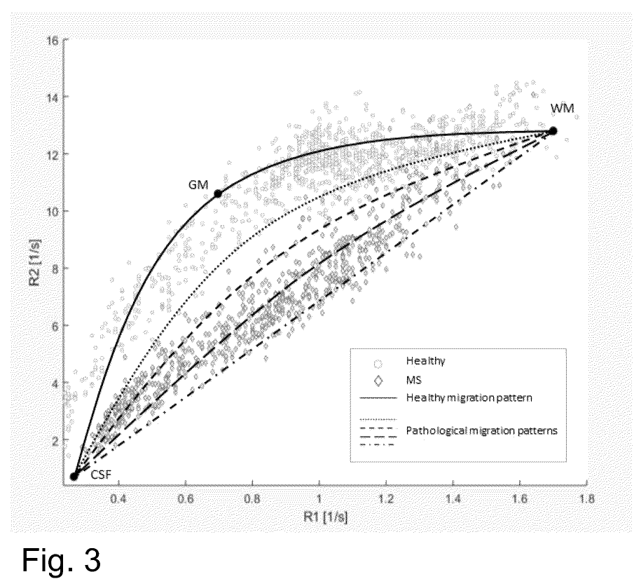

In connection with FIG. 3, the lesion evolution model will be discussed in more detail.

FIG. 3 is an example of a plot of R1 and R2 values, as the plots of FIG. 1. The round points of FIG. 3 represent the R1 and R2 values of voxels of an MR image of a healthy brain tissue. The diamond-shaped points of FIG. 3 represent the R1 and R2 values of voxels of another MR image of a patient suffering from a progressive MS.

From FIG. 1, it is known that the R1 and R2 values of a brain tissue of MS lesion are typically lower than that of a corresponding healthy brain tissue, this is also true in FIG. 3, as most of the round points are in the upper-right part of the plot, while most of the diamond-shaped points are in the lower-left part of the plot.

The lesion evolution model may match the tissue composition to a lesion evolution status. That is, based on the lesion evolution model and a set of predetermined quantitative values, it is possible to determine the lesion evolution status of the tissue having the quantitative values.

The R1 and R2 values of the upper point “WM” in FIG. 3 may be determined to be the first set of quantitative values of the lesion evolution model, representing a healthy brain tissue. The R1 and R2 values of the lower point “CSF” in FIG. 3 may be determined to be the second set of quantitative values of the lesion evolution model, representing a necrotic brain tissue. The initial status is a normal status, e.g., a healthy status, and the final status is an abnormal status, e.g., a necrosis status, or vice versa.

The lesion evolution model may comprise a plurality of predetermined sets of quantitative values. The lesion evolution value of a voxel of an MR image (representing an imaged tissue) may be determined by comparing its quantitative values (e.g., R1 and R2 values) with the plurality of predetermined sets of quantitative values of the lesion evolution model. A set predetermined of quantitative values of the lesion evolution model which is closest to the quantitative values of the voxel may be determined, and the lesion evolution value of the voxel may be determined to be equal to the reference lesion evolution value associated with the determined set of quantitative values of the lesion evolution model.

For example, if the R1 and R2 values of a voxel corresponding to an imaged tissue are 1.5 s⁻¹ and 12.9 s⁻¹, respectively, which are closest to the first set of R1 and R2 values of WM (R1=1.6 s⁻¹ and R2=12.8 s⁻¹), the lesion evolution value of this tissue can be determined to be equal to the reference lesion evolution value associated with the determined first set of quantitative values. In other words, this tissue is considered to be a healthy tissue.

The term “closest” may be interpreted as a minimal deviation between two sets of data. There are various mathematical ways to calculate such deviation between two sets of data, each set comprising at least two elements.

The at least two predetermined sets of quantitative values of the lesion evolution model may be scatter-plotted as at least two lesion evolution points, respectively in a coordinate system, wherein the first and second predetermined set of quantitative values may be scatter-plotted into a first lesion evolution point, e.g., the lesion evolution point “WM” and a second lesion evolution point, e.g., the lesion evolution point “CSF” in the R1-R2 space of FIG. 3.

The method may comprise generating a lesion evolution curve passing through the at least two lesion evolution points one by one in the coordinate system, following an order of lesion evolution from the initial status to the final status, wherein the lesion evolution curve starts from the first lesion evolution point, e.g., the lesion evolution point “WM”, and ends at the second lesion evolution point, e.g., the lesion evolution point “CSF” in the R1-R2 space of FIG. 3. Alternatively, the lesion evolution curve may start from the second lesion evolution point, e.g., the lesion evolution point “CSF”, and end at the first lesion evolution point, e.g., the lesion evolution point “WM” in the R1-R2 space of FIG. 3.

The lesion evolution curve may be generated based on the diamond-shaped points of FIG. 3.

In FIG. 3, the upper solid curved line derived based on the round points illustrates the typical pattern of the healthy tissue. The lower four curved lines are examples of lesion evolution curves illustrating migration patterns. In FIG. 3, the four examples of lesion evolution curves are generated as a parametric curve, with parameters chosen to intersect the WM and CSF clusters formed by the diamond-shaped points of FIG. 3. Although the four examples of lesion evolution curves are different, they all start from the first lesion evolution point “WM”, and ends at the second lesion evolution point “CSF”, or vice versa.

Alternatively, or in combination, a parametric representation describing a relationship between the at least two lesion evolution points in the coordinate system, based on the at least two predetermined sets of quantitative values, may be generated, with or without scatter-plotting the at least two predetermined sets of quantitative values.

The lesion evolution curve may be generated by connecting the at least two lesion evolution points one by one.

The lesion evolution curve may be a continuous line or may comprise a plurality of discontinuous portions. The lesion evolution curve may comprise a straight portion and/or a curved portion.

A portion of the lesion evolution curve between two lesion evolution points may be straight, e.g., a shortest straight line between the two lesion evolution points in the coordinate system.

The portion of the lesion evolution curve between two lesion evolution points may be curved. The exact shape of the curve may be defined by the parametric representation.

Following any one of the four examples of lesion evolution curves of FIG. 3, it can be seen that in the initial status (the first lesion evolution point “WM”), the tissue is healthy white matter as the R1 and R2 values of the tissue indicates so (see Table 1). Then, demyelination and inflammation cause the R1 and R2 values of the MS pathological tissue to decrease, i.e. progressing downwards following any one of the four examples of the lesion evolution curves of FIG. 3. Finally, when the lesion becomes more severe, the quantitative values of physical properties, e.g., R1 and R2, of the tissue arrives at the final status indicating necrosis (the second lesion evolution point “CSF”).

However, if the lesion recovers, it may travel along the lesion evolution curve in the opposite direction. The quantitative values of the physical properties, e.g., R1 and R2, of the tissue may travel from left to right following any of the examples of the lesion evolution curves.

The final status and the initial status may be determined based on different types of lesions. The initial to final status may cover an entire lesion process of health to necrosis, or a recover process. The initial to final status may cover only a part of the entire process.

The predetermined set of quantitative values and/or the combination of physical properties may be different for different types of lesions and/or different body portions. For example, for a brain tissue, it is known that it comprises mainly white matter, grey matter and water. However, a knee portion comprises mainly muscles, bones and cartilage. Naturally, the lesion evolution model for different diseases of different body portions may be very different.

It can be difficult to predict the exact pathological migration pattern, e.g., the diamond-shaped points in FIG. 3 being rather scattered with outliers. Thus, depending on the specific mathematical methods used for designing the lesion evolution model, i.e. determining the predetermined sets of quantitative values of different lesion evolution statuses, the lesion evolution curves may appear differently, e.g., a steeper appearance or a less steep appearance, as the four examples of lesion evolution curves shown in FIG. 3.

Based on the generated lesion evolution curve, a predetermined number of new lesion evolution points may be generated between the first and the second lesion evolution point in the coordinate system. The new lesion evolution points may be points of the lesion evolution curve.

Based on the parametric representation, a predetermined number of new lesion evolution points may be generated between the first and the second lesion evolution point in the coordinate system. Each of the new lesion evolution points corresponds to a new set of quantitative values. The new sets of quantitative values may also fulfill the parametric representation.

For each tissue (a voxel of an MR image presenting the tissue), its lesion evolution value may be determined to be the reference lesion evolution value of the lesion evolution point having a shortest distance to the evolution point of the tissue. A large number of lesion evolution points may improve the accuracy of the method.

For each new set of quantitative values, a new reference lesion evolution value indicating a new status of lesion evolution may be associated. The associated new reference lesion evolution value may be determined based on the generated lesion evolution curve or the generated parametric representation, and the at least two predetermined sets of quantitative values and their associated reference lesion evolution values.

After the generation of the new lesion evolution points, a new lesion evolution curve may be generated in the coordinate system. The new lesion evolution curve may start from the first lesion evolution point, pass through the new lesion evolution points one by one, end at the second lesion evolution point, following an order of lesion evolution from the initial status to the final status. The new lesion evolution curve may start from the second lesion evolution point, pass through the new lesion evolution points one by one, end at the first lesion evolution point, following an order of lesion evolution from the final status to the initial status.

Alternatively, or in combination, after the generation of the new lesion evolution points, an existing lesion evolution curve in the coordinate system, e.g., any of the four examples of the lesion evolution curves of FIG. 3 may be refined using the new lesion evolution points. The refined lesion evolution curve may start from the first lesion evolution point, pass through the new lesion evolution points one by one, end at the second lesion evolution point, following an order of lesion evolution from the initial status to the final status, or in the reverse direction.

The initial status may be a normal status and the final status may be an abnormal status. Alternatively, the initial status may be an abnormal status and the final status may be a normal status.

The generated new lesion evolution points and the at least two lesion evolution points may be evenly positioned along the generated new lesion evolution curve or the refined lesion evolution curve.

The associated reference lesion evolution values of the at least two predetermined sets of quantitative values and of the new sets of quantitative values may form an arithmetic evolution, starting with the first reference lesion evolution value and ending with the second reference lesion evolution value. A position of each reference lesion evolution value of the arithmetic evolution corresponds to a position of its corresponding lesion evolution point along the generated new lesion evolution curve or the refined lesion evolution curve.

For example, the lesion evolution curve may comprise 101 points evenly distributed throughout the line, each being assigned with a reference lesion evolution value being an element of an arithmetic evolution of 101 elements, which are 0, 1, 2, . . . , 98, 99 and 100. The reference lesion evolution value of 0 indicates a healthy status, the reference lesion evolution value of 50 indicates a status of a progressed lesion, and the reference lesion evolution value of 100 indicates a status of total tissue destruction.

Although the lesion evolution curves are used as examples, it is noted that the lesion evolution model may merely comprise the predetermined sets of quantitative values. These predetermined set of quantitative values may be presented in the form of a table, a polynomial function, or a parametric curve for defining the relationship of the predetermined sets of quantitative values.

The method may comprise repeating the step of determining a lesion evolution value for each voxel of the region of interest. Consequently, the lesion evolution value for each voxel of the region of interest may be determined. The lesion evolution value for each voxel of the first MR image may be determined.

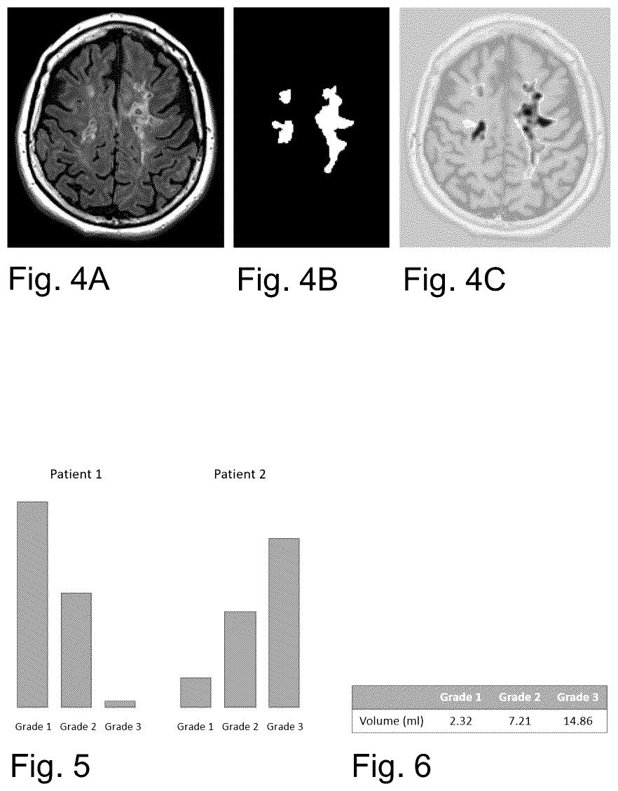

In connection with FIGS. 4A-6 and FIG. 12, the lesion evolution visualization will be discussed in more detail.

FIG. 4A is the same T2FLAIR image as FIG. 2A illustrating a patient's brain tissues. Some of the lesions may be detected by the bright appearances of certain voxels. However, it is not possible to understand the severity of the lesions by analyzing the T2FLAIR image of FIG. 4A.

The method may comprise segmenting a part of the imaged portion. The segmenting may result in a region of interest of the MR image representing the segmented part of the imaged portion. Segmenting or segmentation is a well-known method for analyzing the MR images.

FIG. 4B illustrates a few regions of interest. The region of interest may comprise a small portion of the MR image, comprising voxels representing a part of the imaged portion, e.g., a lesion. The region of interest may be selected by manual segmentation or by automatic segmentation.

The lesion evolution of the portion may be visualized, e.g., by displaying the determined lesion evolution value.

An overlay may be visualized. The voxels of the overlay corresponding to the voxels of the regions of interest may be displayed differently based on the different lesion evolution values of the voxels of the regions of interest.

FIG. 4C is a FLAIR-MASK-Lesion evolution image, visualizing such an overlay, wherein the voxels of the overlay corresponding to the voxels of the regions of interest (FIG. 4B) are displayed in different intensities, based on the lesion evolution values of these voxels of the region of interest. Thus, the severity of lesion for each voxel of the regions of interest are visualized by different intensities of the voxels of the overlay.

An intensity scale may be displayed as a reference for correlating a lesion evolution value to an intensity of the voxels displayed differently.

By analyzing the overlay, it is possible to draw conclusions about the lesion evolution status of the lesions. Comparing with the T2FLAIR image of FIG. 4A, a fast and straightforward understanding of the lesion evolution status of the patient can be concluded based on the overlay of FIG. 4C.

Alternatively, or in combination, the voxels of the overlay corresponding to the voxels of the region of interest may be displayed in different colors based on the lesion evolution values of these voxels of the region of interest. A color scale may be displayed as a reference for correlating a lesion evolution value to a color of the voxels displayed differently.

In the example of FIG. 4C, the T2FLAIR image of FIG. 4A is provided as a background image representing the portion. The background image may be used to provide a view of the anatomy of the imaged portion. Thus, using the background image may facilitate the correlation of lesion status and the anatomy of the imaged portion. The background image may be the first MR image representing the portion, generated based on the single MR sequence, or a contrast weighted image.

Alternatively, using the overlay and the background image is not necessary. Visualizing lesion evolution of the portion may comprise displaying the voxels of the region of interest of the first MR image differently, based on their respective lesion evolution value. In other words, it is possible to visualize the lesion evolution in the first MR image generated based on the single MR sequence, without any overlays.

The method may further comprise for each determined lesion evolution value, calculating a volume size of a partial portion having the status of lesion evolution indicated by said determined lesion evolution value.

The method may further comprise displaying a distribution of the determined lesion evolution value for each voxel of the region of interest, e.g., in a form of histogram.

FIG. 5 are histograms respectively visualizing the determined lesion evolution values for two MS patients.

In the example of FIG. 5, the histogram of Grade 1 representing the number of voxels having a low lesion evolution value (e.g., 0-20) indicating an initial stage of MS lesion evolution. The histogram of Grade 2 representing the number of voxels having an intermediate lesion evolution value (e.g., 21-80) indicating a progressed stage of MS lesion evolution. The histogram of Grade 3 representing the number of voxels having a high lesion evolution value (e.g., 81-100) indicating a final stage of MS lesion evolution.

From the histograms of FIG. 5, it can be seen that patient 1 (the left histogram) has a large lesion load of low lesion evolution values (Grade 1) and a very small lesion load of high lesion evolution values (Grade 3). That is, the MS lesion of patient 1 is primarily of the initial active phase of inflammation and edema. In contrast, patient 2 (the right histogram) has a large lesion load of high lesion evolution values (Grade 3) and a small lesion load of low lesion evolution values (Grade 1). That is, the MS lesions of patient 2 is primarily of a final stage of severe tissue destruction.

The example of FIG. 5 may provide a simplified way of visualizing the lesion evolution status.

The Grades 1-3 of FIG. 5 are merely examples. For example, more than three grades can be used in the histogram. For example, the distribution of each lesion evolution value may be visualized. The histogram may also be normalized, such that a proportion of voxels that having each lesion evolution value may be visualized.

A lesion evolution profile may provide a high-resolution visualization of tissue status. It may be conceptualized as a refined version of the histograms shown in FIG. 5, wherein voxel counts are aggregated over narrower intervals of lesion evolution values. This refinement enables a more detailed and accurate representation of tissue condition compared to the lower resolution example illustrated in FIG. 5.

The lesion evolution profile may comprise an x-axis representing lesion evolution values, and a y-axis representing the corresponding volume size for each lesion evolution value or lesion evolution value range.

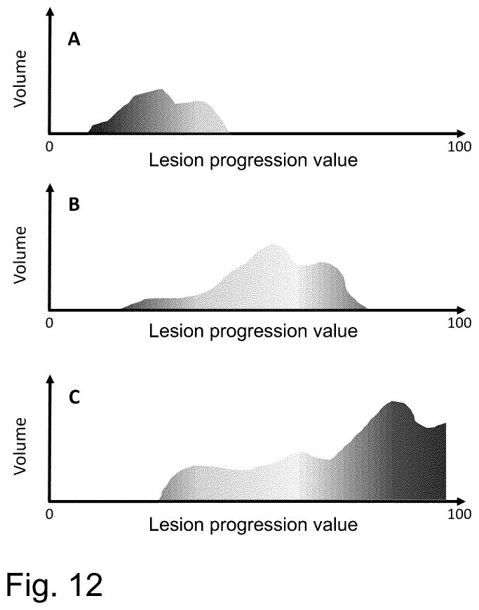

FIG. 12 illustrates three exemplary evolution profiles. In these examples, the x-axis represents the evolution lesion evolution values spanning a range from 0 to 100, and the y-axis represents the volume size for each lesion evolution value or lesion evolution value range within the same range. For example, a lesion evolution value range may be set to 5, 1, or 0.1.

In Example A of FIG. 12, the majority of tissue exhibits relatively low lesion evolution values (less than 50), indicating ongoing inflammation without significant tissue destruction. This suggests that tissue recovery remains possible.

In Example B of FIG. 12, lesion evolution values are distributed across the central portion of the full range (0 to 100), indicating advancing lesion evolution and partial tissue damage, with limited potential for recovery.

In Example C of FIG. 12, most tissue exhibits high lesion evolution values (greater than 50), signifying severe lesion evolution and extensive tissue destruction. This suggests that large portions of the tissue are likely beyond recovery.

As demonstrated in FIG. 12, the lesion evolution profile provides an overall assessment of tissue condition, facilitates rapid and intuitive understanding of the extent of tissue damage and the potential for recovery.

This is advantageous for clinical decision-making and treatment planning.

In addition to assessing the current tissue condition, it is also of interest to evaluate how the condition evolves over time. For example, if a patient has large volumes of tissue destruction and high lesion evolution values, but the condition remains stable overtime, it may suggest that the lesion worsening has been halted and there is no need to adjust the medication.

Accordingly, it would be advantageous to provide a longitudinal visualization of lesion evolution.

When multiple single MR sequences of the same tissue portion are acquired over time, corresponding lesion evolution profiles may be generated to monitor the temporal development of lesion evolution. For example, if the lesion evolution profiles shown in Examples A to C of FIG. 12 were obtained based on three successive acquisitions, it would indicate that the lesion evolution advances over time.

The method may further comprise acquiring, at a second time point, a second single MR sequence of the portion comprising quantitative information of the portion. The second single MR sequence may be processed as for the first single MR sequence.

The method may further comprise generating a second MR image representing the portion, based on the second single MR sequence, wherein the second MR image comprises a second region of interest, wherein both the region of interest and the second region of interest correspond to a same part of the portion; for a voxel of the second region of interest, determining a second lesion evolution value, based on quantitative values of said voxel of the second region of interest and the lesion evolution model; and determining a change of lesion evolution of the portion from the first time point to the second time point, based on the determined second lesion evolution value, and the lesion evolution value of its corresponding voxel of the region of interest.

The method may further comprise generating a second lesion evolution profile, comprising: repeating the step of determining the second lesion evolution value for each voxel of the second region of interest; for each determined second lesion evolution value, calculating a volume size of a partial portion having the status of lesion evolution indicated by said determined second lesion evolution value; displaying a distribution of determined lesion evolution values for each voxel of the region of interest as a second lesion evolution profile in the coordinate system.

The method may further comprise displaying the change of lesion evolution of the portion from the first time point to the second time point by subtracting the first lesion evolution profile from the second lesion evolution profile.

To assess lesion evolution over time, the difference between two lesion evolution profiles, obtained from two single MR sequences acquired at two distinct time points, may be computed by subtracting the second lesion evolution profile from the first. This enables visualization of the change in lesion evolution profiles between the two time points.

The resulting change in lesion evolution may be visualized within the same coordinate system, with the y-axis extended to include negative values. Positive values along the y-axis indicate further lesion evolution, while negative values reflect tissue recovery.

Image registration between the two MR scans may be required to ensure accurate alignment for longitudinal analysis.

Additionally, the computed changes in lesion evolution may be visualized as an overlay, for example, as shown in FIGS. 4A to 4C. This allows identification of recovering lesions and differentiation between chronic active and chronic inactive lesions. Such differentiation serves as an important biomarker, as chronic active lesions are a key driver of overall disease evolution in MS.

FIG. 6 visualize the volume sizes (ml) of the tissues of different lesion evolution stages, i.e. Grade 1, 2, and 3. The volume size may be calculated based on the absolute volume size for each voxel, and the number of the voxels having the same lesion evolution value.

The volume sizes may be in the form of percentage (%) indicating a percentage of the tissue of different lesion evolution stages.

Other information may be further derived based on the determined lesion evolution values, e.g., the distribution of the determined lesion evolution values.

The derived information may be presented as texts, e.g., in a table as FIG. 6, or as graphics. This may provide a quick overview of the lesion evolution status.

The change of status of lesion evolution of the portion may be determined by comparing the determined lesion evolution values, or any of the generated overlays such as FIG. 4C, the derived histogram such as FIG. 5 or the derived volume size such as FIG. 6. A quick overview of the change of status of lesion evolution, e.g., the lesion is recovered or progressed, may be achieved.

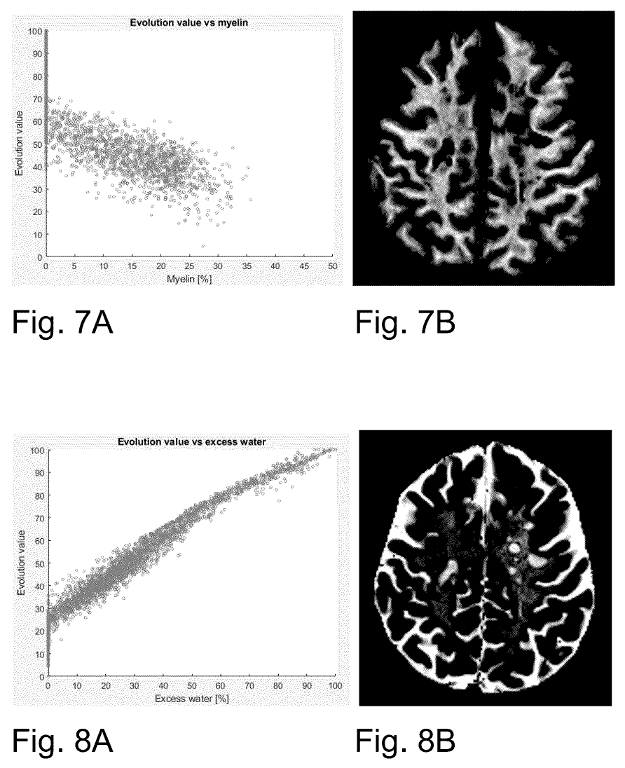

In connection with FIGS. 7A-10B, the correlation between lesion evolution values (0-100) and other measurements will be discussed in more detail. In these figures, the lesion evolution value being 0 refers to a healthy status, and the lesion evolution value being 100 refers to a narcosis status due to MS.

FIG. 7A is a plot for illustrating the correlation between each lesion evolution value determined by the method and the corresponding myelin content (%) of the corresponding voxel. FIG. 7B is a myelin graph visualizing the myelin of the same imaged portion of FIG. 7A.

From FIG. 7A, a myelin degeneration can be seen as most of the data points have a myelin content of less than 30%. Further, a higher concentration of myelin correlates with a minor lesion evolution value. Thus, the lesion evolution value indeed confirms a further progressed MS lesion evolution. From FIG. 7A, it can also be seen that the complete myelin destruction (nearly 0%) appears when the lesion evolution values are larger than approximately 70. The lesion evolution value determined by the method and the lesion evolution model show a good correlation between the lesion evolution values and the myelin content of the imaged portion.

FIG. 8A is a plot for illustrating the correlation between each lesion evolution value determined by the method and the corresponding excess water content (%) of the corresponding voxel. FIG. 8B is a CSF graph visualizing the excess water content of the same imaged portion of FIG. 8A.

From FIG. 8A, it can be seen that the CSF concentration almost linearly increases along with the increase of the lesion evolution values. That is, the larger the lesion evolution value, the higher content of the excess water. This agrees with the ground theory, i.e. the high water concentration indicating a widening of the extracellular space due to tissue destruction. From FIG. 8A, it can also be concluded that when the lesion evolution value is less than 30, there is only a tiny amount of excess water or no excess water exists.

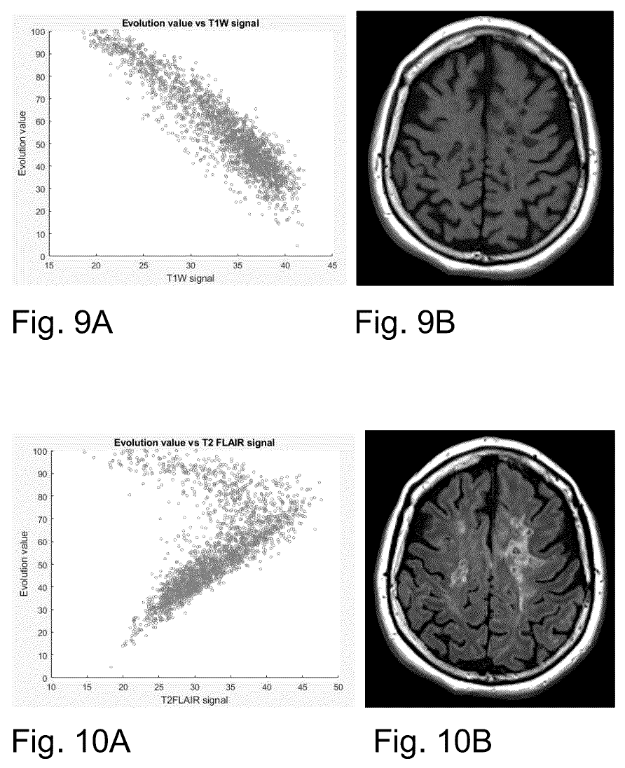

FIG. 9A is a plot for illustrating the correlation between each lesion evolution value determined by the method and the signal intensity of the corresponding voxel of the T1W image. FIG. 9B is the T1W image of the same imaged portion of FIG. 9A.

The MS lesions are isointense or hypointense (dark, i.e. low signal intensity compared to surrounding tissue) in the T1W image of FIG. 9B. The hypointense black holes of the T1W image are associated with severe inflammation and tissue destruction. This relationship can be clearly seen in FIG. 9A as the signal intensity decreases when the lesion evolution value increases.

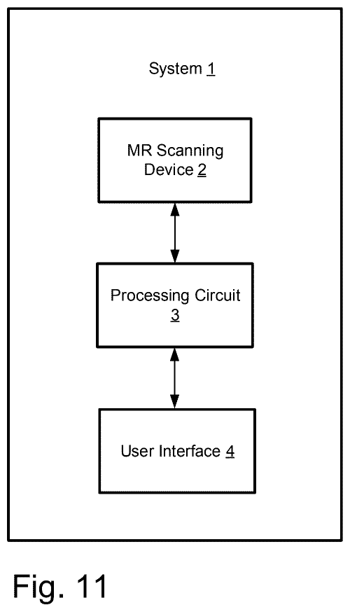

FIG. 10A is a plot for illustrating the correlation between each lesion evolution value determined by the method and the signal intensity of the corresponding voxel of the T2FLAIR image.

FIG. 10B is the T2FLAIR image of the same imaged portion of FIG. 10A. The MS lesions are hyperintense (bright, i.e. high signal intensity compared to surrounding tissue) or hypointense (due to the high water concentration) in the T2FLAIR image of FIG. 10B.

As shown in FIG. 10A, the signal intensity increases along with the increase of the lesion evolution value until reaching about 70. However, in FIG. 10A, the signal intensity drastically decreased after the lesion evolution value continues increases. As discussed in combination with FIG. 2A, due to that the water concentration increases too much, the signal of the water is suppressed by the inversion pulse causing a reduced signal intensity. Thus, the V shaped of FIG. 10A perfectly match the theory.

Thus, the validity of the invention can be demonstrated by the examples of FIGS. 7A-10B.

The present invention can provide not only the information of the T1W and T2FLAIR images, but also a more detailed and quantified information of the lesion evolution. The detailed and quantified information of the lesion evolution approach may be presented in a variety of ways for different usages. A great advantage is that the invention is based on quantitative information, making it possible to study the lesion evolution changes over time for an individual patient. Such quantified lesion evolution may be used as an indicator, e.g., for screening for diseases, for predicting the tissue destructions, etc.

1. A method of quantifying lesion evolution, comprising:
acquiring, at a first time point, by a magnetic resonance, MR, scanning device, a single MR sequence of a portion of a body, the single MR sequence comprising quantitative information of the portion;
generating, by a processing circuit, a first MR image representing the portion, based on the single MR sequence, wherein each voxel of the first MR image represents a corresponding volume of the portion;
providing a lesion evolution model,
wherein the lesion evolution model comprises at least two predetermined sets of quantitative values, comprising a first predetermined set of quantitative values representing the portion at an initial status of lesion evolution, and a second predetermined set of quantitative values representing the portion at a final status of lesion evolution, and
wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution;
for a voxel of a region of interest of the first MR image, determining, by the processing circuit, a lesion evolution value of said voxel, indicating a status of lesion evolution at the first time point, based on a comparison between quantitative values of the voxel and the lesion evolution model.

2. The method of claim 1, wherein the quantitative information comprises information of at least two among the physical properties: a longitudinal relaxation rate R1, a transverse relaxation rate R2, a longitudinal relaxation time T1, a transverse relaxation time T2, and a Proton Density, PD.

3. The method of claim 1, wherein the step of determining a lesion evolution value of said voxel comprises:
among the at least two predetermined sets of quantitative values of the lesion evolution model, determining a set of quantitative values being closest to the quantitative values of the voxel, and
determining the lesion evolution value of the voxel to be equal to the reference lesion evolution value associated with the determined set of quantitative values.

4. The method of claim 1, further comprising:
for at least one of the at least two predetermined sets of quantitative values, calculating the reference lesion evolution value to be associated, based on said predetermined set of quantitative values.

5. The method of claim 4, wherein said predetermined set of quantitative values comprises a value of the longitudinal relaxation rate R1 and a value of the transverse relaxation rate R2.

6. The method of claim 5, further comprising:
calculating the lesion evolution value to be associated by
Lesion Evolution Value=norm(R1)*norm (R2),
wherein Lesion Evolution Value refers to the reference lesion evolution value to be associated with said set of quantitative values, R1 and R2 respectively refer to the values of the longitudinal relaxation rate R1 and the transverse relaxation rate R2 of said set of quantitative values, norm refers to a norm function.

7. The method of claim 1, further comprising:
scatter-plotting the at least two predetermined sets of quantitative values into at least two lesion evolution points, respectively, in a coordinate system, wherein the first and second predetermined set of quantitative values are scatter-plotted into a first lesion evolution point and a second lesion evolution point, respectively.

8. The method of claim 7, further comprising:
generating a lesion evolution curve passing through the at least two lesion evolution points one by one in the coordinate system, following an order of lesion evolution from the initial status to the final status, wherein the lesion evolution curve starts from the first lesion evolution point, and ends at the second lesion evolution point, or following an order of lesion evolution from the final status to the initial status, wherein the lesion evolution curve starts from the second lesion evolution point, and ends at the first lesion evolution point; or
generating a parametric representation describing a relationship between the at least two lesion evolution points in the coordinate system, based on the at least two predetermined sets of quantitative values.

9. The method of claim 8, further comprising:
creating a predetermined number of new lesion evolution points between the first and the second lesion evolution point in the coordinate system, based on the generated lesion evolution curve or the generated parametric representation, wherein each of the new lesion evolution points corresponds to a new set of quantitative values.

10. The method of claim 9, further comprising:
for each new set of quantitative values, associating a new reference lesion evolution value indicating a new status of lesion evolution.

11. The method of claim 10, wherein for each new set of quantitative values, the associated new reference lesion evolution value is determined based on the generated lesion evolution curve or the generated parametric representation, and the at least two predetermined sets of quantitative values and their associated reference lesion evolution values.

12. The method of claim 9, further comprising:
generating a new lesion evolution curve or refining an existing lesion evolution curve in the coordinate system, which passes through the at least two lesion evolution points and the generated new lesion evolution points one by one, following an order of lesion evolution from the initial status to the final status, wherein the generated new lesion evolution curve or the refined lesion evolution curve starts from the first lesion evolution point, and ends at the second lesion evolution point, or
following an order of lesion evolution from the final status to the initial status, wherein the generated new lesion evolution curve or the refined lesion evolution curve starts from the second lesion evolution point, and ends at the first lesion evolution point.

13. The method of claim 12, wherein the new lesion evolution points and the at least two lesion evolution points are evenly positioned along the generated new lesion evolution curve or the refined lesion evolution curve; and
wherein the associated reference lesion evolution values of the at least two predetermined sets of quantitative values and of the new sets of quantitative values form an arithmetic evolution;
wherein the arithmetic evolution starts with the first reference lesion evolution value and ends with the second reference lesion evolution value; and
wherein a position of each reference lesion evolution value of the arithmetic evolution corresponds to a position of its corresponding lesion evolution point along the generated new lesion evolution curve or the refined lesion evolution curve.

14. The method of claim 8, wherein the lesion evolution curve comprises a straight portion.

15. The method of claim 1, wherein the initial status is a normal status and the final status is an abnormal status; or
wherein the initial status is an abnormal status and the final status is a normal status.

16. The method of claim 1, further comprising:
visualizing lesion evolution of the portion, comprising:
displaying, by a user interface, the determined lesion evolution value.

17. The method of claim 1, further comprising:
repeating the step of determining a lesion evolution value for each voxel of the region of interest.

18. The method of claim 17, further comprising:
for each determined lesion evolution value, calculating a volume size of a partial portion having the status of lesion evolution indicated by said determined lesion evolution value; and
displaying a distribution of determined lesion evolution values for each voxel of the region of interest in a form of histogram.

19. The method of claim 18, wherein the histogram is a lesion evolution profile in a coordinate system;
wherein an x-axis of the coordinate system represents lesion evolution values, and a y-axis of the coordinate system represents a volume size for each lesion evolution value or lesion evolution value range.

20. The method of claim 1, further comprising:
visualizing lesion evolution of the portion, comprising:
displaying the first MR image, wherein the voxels of the region of interest are displayed differently, based on their respective lesion evolution value.

21. The method of claim 1, further comprising:
visualizing lesion evolution of the portion, comprising:
displaying the first MR image or a different MR image representing the portion as a background image; and
displaying an overlay to the background image, wherein voxels of the overlay corresponding to the voxels of the region of interest are displayed differently, based on the lesion evolution values of the voxels of the region of interest.

22. The method of claim 20, further comprising:
the voxels displayed differently are displayed in different colors and/or intensities.

23. The method of claim 22, further comprising:
displaying a color scale as a reference for visualizing the lesion evolution value for each displayed color of the voxels displayed differently; and/or
displaying an intensity scale as a reference for visualizing the lesion evolution value for each displayed intensity of the voxels displayed differently.

24. The method of claim 1, further comprising:
acquiring, at a second time point, a second single MR sequence of the portion comprising quantitative information of the portion;
generating a second MR image representing the portion, based on the second single MR sequence, wherein the second MR image comprises a second region of interest, wherein both the region of interest and the second region of interest correspond to a same part of the portion;
for a voxel of the second region of interest, determining a second lesion evolution value, based on quantitative values of said voxel of the second region of interest and the lesion evolution model; and
determining a change of lesion evolution of the portion from the first time point to the second time point, based on the determined second lesion evolution value, and the lesion evolution value of its corresponding voxel of the region of interest.

25. The method of claim 24, wherein the step of determining a change of lesion evolution of the portion from the first time point to the second time point comprises:
repeating the step of determining a lesion evolution value for each voxel of the region of interest;
for each determined lesion evolution value, calculating a volume size of a partial portion having the status of lesion evolution indicated by said determined lesion evolution value;
displaying a distribution of determined lesion evolution values for each voxel of the region of interest as a first lesion evolution profile in a coordinate system;
wherein an x-axis of the coordinate system represents lesion evolution values, and a y-axis of the coordinate system represents a volume size for each lesion evolution value or lesion evolution value range;
repeating the step of determining a second lesion evolution value for each voxel of the second region of interest;
for each determined second lesion evolution value, calculating a volume size of a partial portion having the status of lesion evolution indicated by said determined second lesion evolution value;
displaying a distribution of determined lesion evolution values for each voxel of the region of interest as a second lesion evolution profile in the coordinate system;
displaying the change of lesion evolution of the portion from the first time point to the second time point by subtracting the first lesion evolution profile from the second lesion evolution profile.

26. The method of claim 1, wherein the portion of the body comprises a head or any part of a spine.

27. The method of claim 1, wherein the lesion comprises a tissue abnormality.

28. A system for quantifying lesion evolution, comprising a magnetic resonance, MR, scanning device configured to:
acquire, at a first time point, a single MR sequence of a portion of a body, the single MR sequence comprising quantitative information of the portion;
the system for quantifying lesion evolution comprising a processing circuit configured to:
generate a first MR image representing the portion, based on the single MR sequence, wherein each voxel of the first MR image represents a corresponding volume of the portion; and
determine a lesion evolution value indicating a status of lesion evolution for a voxel of a region of interest of the first MR image, based on a comparison between quantitative values of the voxel and a lesion evolution model;
wherein the lesion evolution model comprises at least two predetermined sets of quantitative values, comprising a first predetermined set of quantitative values representing the portion at an initial status of lesion evolution, and a second predetermined set of quantitative values representing the portion at a final status of lesion evolution;
wherein each of the at least two predetermined sets of quantitative values is associated with a reference lesion evolution value indicating a status of lesion evolution, the first predetermined set of quantitative values is associated with a first reference lesion evolution value indicating the initial status of lesion evolution, and the second predetermined set of quantitative values is associated with a second reference lesion evolution value indicating the final status of lesion evolution.

29. A non-transitory computer readable recording medium having computer readable program code recorded thereon which when executed on a device having processing capability is configured to perform the method of claim 1.
